(19) United States
(12) Patent Application Publication
(10) Pub. No.: US 2005/0195944 A1
(43) Pub. Date: Sep. 8, 2005

(54) Patient support device for computer tomography

(76) Inventors: Frank Bartels, Seybothenreuth (DE); Stefan Leidenberger, Effeltrich (DE); Paul Weidner, Pressath (DE)

Correspondence Address: BRINKS HOFER GILSON & LIONE, P.O. BOX 10395, CHICAGO, IL, 60610, US

(21) Appl. No.: 10/974,069

(22) Filed: Oct. 27, 2004

(30) Foreign Application Priority Data
Oct. 31, 2003 (DE) 103 50 898.8

(51) Int. Cl. G21K 1/12; H05G 1/60; A61B 6/00; G01N 23/00; H05G 1/02

(52) U.S. Cl. 378/195; 378/20

(57) ABSTRACT

A patient support device for a computer tomography system is provided. The computer tomography system has a gantry which is tiltable about a horizontal axis. The patient support device configured to support a patient bed comprises a C-shaped telescopic arm connected to the gantry, and a patient bed guide configured to carry the patient bed is connected to the C-shaped telescopic arm such that the patient bed is moved by an extension or retraction movement of the telescopic arm relative to the gantry.

### BACKGROUND

The invention relates, in general, to clinical patient tables or support units, and more particularly to a patient support device for a computer tomography system.

A computer tomography system, also hereafter abbreviated to CT, is used to create three-dimensional or sectional images of a body of a patient to be examined. The computer tomography system features a gantry within which an x-ray source rotates on a circular path. Diametrically opposite the x-ray source on the same circular path, an x-ray detector rotates. The x-ray detector can detect x-rays transmitted by the x-ray source. The patient to be examined is positioned in a center of the rotational movement of the x-ray source and the x-ray image detector, so that the transmitted x-rays may pass through the patient.

The CT detects as raw image data two-dimensional projections of the body placed in the gantry as x-ray photographs. A direction of projection corresponds to the relevant and relative angle of rotation of the x-ray source and the x-ray detector.

A computer system computes sectional images or three-dimensional image data from the two-dimensional raw image data. The quality of the image data depends on the positioning and stable position of the patient in the gantry. Movements of the patient adversely affect the image quality and should be avoided or limited by a stable patient support device or unit. Typically, depending on the purpose of the examination, the gantry is tilted around a horizontal axis to take the x-ray images. The gantry tilting tilts a position of the axis of rotation of the x-ray source and x-ray detector in relation to a longitudinal axis of the patient to be examined. As such, the patient support device should keep the longitudinal axis of the patient lying on it stable regardless of the tilt direction of the gantry.

In addition, depending on the purpose of the examination, a requirement to change the position of the patient may be justified so that the patient can be examined with either the head or the legs being raised. As such, the patient support device tilts the patient so that their longitudinal axis is tilted upwards or downwards in relation to the axis of rotation of the x-ray source and image detector.

In addition, the patient support device is desirably configured to ensure good accessibility and thereby allow the patient lying thereon to be well looked after and thereby for the patient to be transferred to or from the patient support device with minimal problems or difficulties. In order to facilitate the transfer of the patient to or from the patient support device, the patient support device may be height-adjustable.

U.S. Pat. No. 6,070,281 discloses a patient support device of which the orientation table is arranged on a movable C-arm so that the patient support device may in turn be moved. The C-arm is supported centrally below a table. The height of the table is adjusted by being able to move the C-arm back and forth on the C-arm support. An arrangement of the support of the C-arm below the table restricts access to it.

DE 196 31 896 discloses a patient support device on which the patient table rests on a pedestal. The pedestal is arranged centrally below the patient table. The table is supported on the pedestal so as to be tilted around two horizontal axes, the two axes being parallel to one another. An arrangement of the pedestal below the tabletop restricts the accessibility of the table.

### OBJECT AND SUMMARY

The present invention is defined by the appended claims. This description summarizes some aspects of the present embodiments and should not be used to limit the claims.

One optional object is to specify a patient support device for a CT which, independently of the angle of tilt of the CT gantry, may guarantee a stable position of the patient lying thereon and at the same time may be accessible with minimal restrictions.

Another object is to specify a patient support device for a computer tomography system which may include a gantry which can be tilted around a horizontal axis, where the patient support device is embodied to carry a patient bed. The patient support device may feature a telescopic C-arm which may be embodied to carry the patient bed, and which can be connected to the gantry in such a way that the patient bed is moved in a plane lying perpendicular to the horizontal axis by extending or retracting the telescopic arm relative to the gantry.

An arrangement of the patient support device on the gantry instead of on a pedestal or foot arranged under the patient bed may achieve substantially optimum accessibility of the patient bed or of the patient lying thereon. In order, to achieve a substantially consistent positioning of the patient, the patient bed can be kept stable by extending or retracting the telescopic arm. A tilting movement of the gantry can be compensated for by moving the telescopic arm such that the patient bed is moved relative to the gantry but in the opposite direction. A point of connection between the gantry and the patient support device may describe a circular path for tilting movements of the gantry, keeping the patient bed stable as supported by the C-shape of the telescopic arm, in that the patient bed can also be moved by the telescopic arm on a circular path.

Accommodating the patient support device on the gantry and thereby being as close as possible to the gantry may also have an effect of providing a stable support for the patient bed and may reduce a patient bed bending as a result of a weight of the patient in the area of the gantry.

In an advantageous feature, the patient support device may feature a control device which is connected to the gantry in such a way that to receive a signal depending on the angle of inclination of the gantry as an input signal and which is configured so that, depending on this signal, the control device may generate a control signal and may issue or transmit the signal to the telescopic arm which can be extended or retracted, depending on the reception of this control signal. The control device can thus be used to keep the selected patient bed stable substantially independent of tilting movements of the gantry. With a suitable mechanical layout of the patient support device, the control device may control the telescopic arm such that the patient bed is moved in substantially the opposite direction to the tilting movement of the gantry.

In a further embodiment, the patient support device features a height adjustment device which is connected to the telescopic arm such that it can adjust its height. This produces more flexible options for positioning the patient. In addition, adjustment of the patient bed to a suitable height makes it easier to transfer the patient to and from the bed.

In a further advantageous feature, the patient support device may be configured so that a radius of the C-shaped bend in the telescopic arm corresponds to the radius of the path on which the telescopic arm is moved through a tilting movement of the gantry. As such, the radius of the telescopic arm movement may depend on a point of application or connection of the patient support device on the gantry. The matching of the radii may provide that the position of the patient can be kept independent of tilting movements of the gantry by extending or retracting the telescopic arm.

Illustrative and exemplary embodiments of the invention are described in further detail below with reference to and in conjunction with the figures.

### BRIEF DESCRIPTION OF THE DRAWINGS

FIG. 1 is a schematic perspective view of one embodiment of a CT gantry with a patient support device,

FIG. 2 is a schematic side view of the CT gantry with the patient support device of FIG. 1; and

FIG. 3 is a schematic side view of the CT gantry and the patient support device of FIG. 1 with a control device.

### DETAILED DESCRIPTION OF DRAWINGS

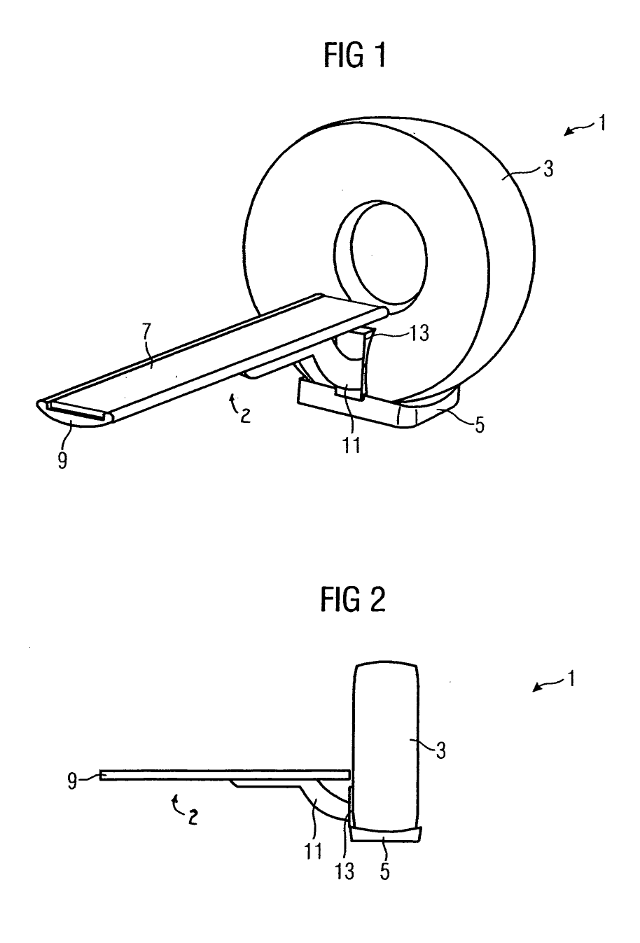

FIG. 1 shows a CT device 1 with patient support device 2. Of the CT 1, only the gantry 3 and the foot 5 supporting the gantry 3 are shown, further details being omitted for reasons of clarity. Within the gantry 3, an x-ray source (not shown) and an x-ray image detector (not shown) may move diametrically opposite each other on a circular path. A center of the circular path is located in a central opening of the gantry 3, into which the patient to be examined is inserted. The gantry 3 is not tilted in the illustration but is oriented vertically.

The patient support device 2 comprises a patient bed 7 which can be inserted into the opening of the gantry 3 so a patient lying thereon can be suitably positioned to allow CT raw images to be taken. The patient bed 7 is supportingly held by a bed guide 9 in which the patient bed 7 can be moved in a longitudinal direction. The patient bed 7 can further be fixed to the bed guide 9 or just be rested thereon.

The bed guide 9 is connected to a C-shaped telescopic arm 11. Extending or retracting the telescopic arm 11 may move the bed guide 9 relatively to the gantry 3. Because of the C-shape of the telescopic arm 11, a circular path of the telescopic arm 11 may be obtained in this arrangement. The telescopic arm 11 is arranged on a lengthwise end of the bed guide 9 such that a space underneath the bed guide 9 may remain free. This free space may increase the accessibility of the patient support device 2 to medical personnel when looking after or transferring the patient onto the patient bed 7.

The telescopic arm 11 may be connected or affixed to the gantry 3, so when the gantry is tilted 3 the telescopic arm 11 also moves along with the gantry 3. The movement of the telescopic arm 11 may describe a circular path when the gantry 3 is tilted. The radius of the circular path depends on the point of application of the telescopic arm 11 to the gantry 3 as well as on the horizontal tilt axis of the gantry 3. In one embodiment, the radius of the C-shape or arc of the telescopic arm 11 may be chosen to be the same as the radius of the circular path of the telescopic arm 11 for tilting movements of the gantry 3.

The telescopic arm 11 may be accommodated on the gantry 3 via a height adjustment device 13 which may allow an adjustment of the height of the telescopic arm 11 and thereby of the patient bed 7. This height adjustment device 13 may extend options for positioning the patient for both taking CT raw data images and for transferring or other desirable activities involving the patient. Adjusting the height of the telescopic arm 11 via the height adjustment device 13 may change the radius of the circular path which describes the movement of the telescopic arm 11 on the basis of tilting movements of the gantry 3.

If the height of the telescopic arm 11 is selected using the height adjustment device 13 so that the radii of the telescopic arm's movement matches tilting movements of the gantry 3 and of the telescopic arm C-shaped bend, then keeping the patient support device 2 stable independently of tilting of the gantry 3 is substantially simple to achieve. As such, extending or retracting the telescopic arm 11 in the opposite direction of the tilting movement of the gantry 3 may be substantially sufficient, where the patient bed 7 is moved in the opposing direction to the tilting movement. Regardless of tilting movements of the gantry 3, the telescopic arm 11 can also be used to change the position of the patient lying on the patient bed 7 so that, for example, he or she can be moved into the gantry 3 with the head or the legs higher than the rest of the patient body.

FIG. 2 shows a schematic side view of the arrangement of CT 1 and of the patient support device 2 as shown in the previous FIG. 1. The gantry 3 may rest vertically oriented on the foot 5. The telescopic arm 11 may be extended to the point at which the bed guide 9 is horizontally oriented and thereby aligned at right angles to the gantry 3. Any tilting movement of the gantry 3 would occur in the selected illustration around a horizontal axis which is oriented at right angles to the plane of the drawing.

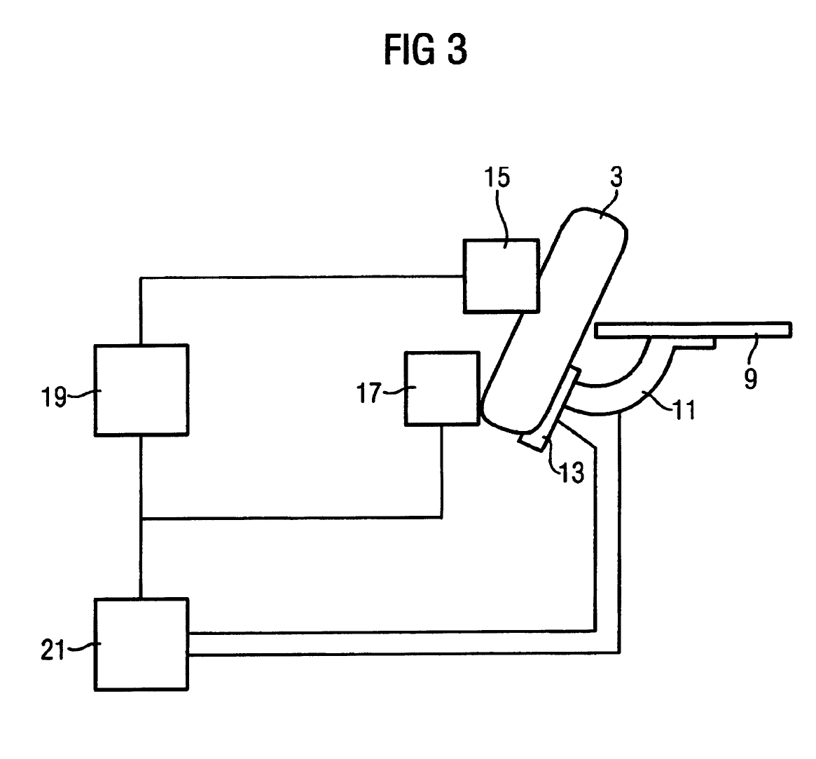

FIG. 3 shows the gantry 3 with the patient support device 2 and an associated control device or unit 21 in a schematic side view. The gantry 3 may be connected to a tilt angle motor 15 which may be activated by a tilt angle control 19. In the FIG. 3 illustration, the gantry 3 may be tilted by the tilt angle motor 15 in the clockwise direction.

The tilt angle control 19 may be connected to the control device 21. The tilt angle control 19 may generate an output signal which represents a current angle of inclination of the gantry 3 and which is received by the control device 21 as an input signal. In addition, a tilt angle recorder 17 may be arranged in the area of the gantry 3 which detects the gantry tilt angle. The detection can for example be done optically by monitoring a visual angular scale or electrically by a potentiometric angle recorder.

The control device 21 may generate control signals as output signals for activating the telescopic arm 11 as well as the height adjustment device 13. The control device 21 may thus control the position and orientation of the patient bed 7 through control signals, and can be used to move the patient bed 7 into a position that can be specified by medical personnel.

In addition, the control device 21 can also be used to keep the position of patient bed 7 stable independent of movements of the gantry 3. As such, the control device 21 may first activate the height adjustment device 13 so that the connection point between the telescopic arm 11 and gantry 3 is at such a height that the radius of the C-arc of the telescopic arm 11 may match the radius of the telescopic arm circular movements as a result of tilting movements of the gantry 3. The position of the patient bed 7 now reached and thus of a patient lying on it can be kept stable, by the telescopic arm 11 being extended or retracted in the opposite direction to a tilting movement of the gantry 3. The control device 21 may receive information about the tilting movement by receiving the output signal either from the tilt angle control 19 or from the tilt angle recorder 17.

In an advantageous feature, the control device 21 may have a program which allows the position of the patient bed 7 to be kept stable independent of the height set by the height adjustment device 13. As such, the program may use graphs for each initial position of the patient bed 7, on the basis of which as a result of changes in angle of inclination corresponding control signals for the height adjustment device 13 and the telescopic arm 11 are generated. The graphs may depend on the geometry of the gantry 3 as well as that of the telescopic arm 11 and of the height adjustment device 13, and can either be supplied by the manufacturer of the patient support device 2 or be recorded on commissioning through a calibration process.

1. A patient support device for a computer tomography system, the computer tomography system having a gantry which is tiltable about a horizontal axis, the patient support device configured to support a patient bed, the patient support device comprising:
a C-shaped telescopic arm connected to the gantry; and
a patient bed guide configured to carry the patient bed, the patient bed guide connected to the C-shaped telescopic arm such that the patient bed is moved by an extension or retraction movement of the telescopic arm relative to the gantry.

2. The Patient support device in accordance with claim 1, further comprising:
a control device communicatively connected with the gantry to receive a signal depending on an angle of inclination of the gantry as an input signal, the control device operable to generate a control signal depending on the received input signal for communication to the telescopic arm,
wherein the telescopic arm is extended or retracted based on the control signal.

3. The Patient support device in accordance with claim 1, further comprising a height adjustment device connected with the telescopic arm to adjust a height of the telescopic arm in relation to the gantry.

4. The Patient support device in accordance with claim 2, further comprising a height adjustment device connected with the telescopic arm to adjust a height of the telescopic arm in relation to the gantry.

5. The Patient support device in accordance with claim 1, wherein a radius of the C-shaped bend of the C-shaped telescopic arm corresponds to a radius of a path on which the telescopic arm is moved by a tilting movement of the gantry.

6. The Patient support device in accordance with claim 2, wherein a radius of the C-shaped bend of the C-shaped telescopic arm corresponds to a radius of a path on which the telescopic arm is moved by a tilting movement of the gantry.

7. The Patient support device in accordance with claim 3, wherein a radius of the C-shaped bend of the C-shaped telescopic arm corresponds to a radius of a path on which the telescopic arm is moved by a tilting movement of the gantry.

8. A computer tomography system comprising:
a gantry tiltable about a horizontal axis; and
a patient support device having a C-shaped telescopic arm, the C-shaped telescopic arm connects the patient support device with the gantry; and a patient bed guide configured to carry a patient bed is connected to the C-shaped telescopic arm such that the patient bed is moved relative to the gantry by an extension or retraction movement of the telescopic arm.

9. The computer tomography system in accordance with claim 8, further comprising:
a control device communicatively connected with the gantry to receive a signal depending on an angle of inclination of the gantry as an input signal, the control device operable to generate a control signal depending on the received input signal for communication to the telescopic arm,
wherein the telescopic arm is extended or retracted based on the received control signal.

10. The computer tomography system in accordance with claim 8, further comprising a height adjustment device connected with the telescopic arm to adjust a height of the telescopic arm in relation to the gantry.

11. The computer tomography system in accordance with claim 9, further comprising a height adjustment device connected with the telescopic arm to adjust a height of the telescopic arm in relation to the gantry.

12. The computer tomography system in accordance with claim 8, wherein a radius of the C-shaped bend of the C-shaped telescopic arm corresponds to a radius of a path on which the telescopic arm is moved by a tilting movement of the gantry.

13. The computer tomography system in accordance with claim 9, wherein a radius of the C-shaped bend of the C-shaped telescopic arm corresponds to a radius of a path on which the telescopic arm is moved by a tilting movement of the gantry.

14. The computer tomography system in accordance with claim 10, wherein a radius of the C-shaped bend of the C-shaped telescopic arm corresponds to a radius of a path on which the telescopic arm is moved by a tilting movement of the gantry.

15. A patient support device for a computer tomography system, the computer tomography system having a gantry which is tiltable about a horizontal axis, the patient support device comprising:
a telescopic arm connected to the gantry; and
a patient bed connected with the telescopic arm such that the patient bed is moved by an extension or retraction movement of the telescopic arm relative to the gantry.
